(19) United States
(12) Patent Application Publication
(10) Pub. No.: US 2008/0318897 A1
(43) Pub. Date: Dec. 25, 2008

(54) Composition Containing Swelling Agent Composed of Swellable Water-Soluble Polysaccharide, and Food, Anti-Obesity Agent and Constipation Alleviator Each Containing the Composition

(75) Inventors: Yuji Uzuhashi, Nagano (JP); Masayuki Nakamura, Nagano (JP)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(73) Assignee: INA FOOD INDUSTRY CO., LTD., Nagano (JP)

(21) Appl. No.: 11/573,298

(22) PCT Filed: Aug. 5, 2005
(86) PCT No.: PCT/JP2005/014383

§ 371 Date: Jul. 22, 2008

(30) Foreign Application Priority Data
Aug. 6, 2004 (JP) 2004-231504

(51) Int. Cl. A61K 31/715; A61P 3/04; A61P 1/10; A23L 1/48

(52) U.S. Cl. 514/54; 426/658

(57) ABSTRACT

A composition containing a swelling agent is provided, which less burdens the stomach and intestine even after excessive intake. A food, anti-obesity agent and constipation alleviator each containing the composition is also provided.

[Means for Solution] A composition comprises a swelling agent composed of a water-soluble polysaccharide swellable in Japanese Pharmacopoeia first fluid or Japanese Pharmacopoeia second fluid and water; and a swell inhibitor operative to control the swell of the swelling agent. The swelling agent and the swell inhibitor are mixed in the state of solution, unified and then dried.

### TECHNICAL FIELD

The present invention relates to a composition comprising a swelling agent composed of a water-soluble polysaccharide swellable in Japanese Pharmacopoeia first fluid or Japanese Pharmacopoeia second fluid and water. And the present invention also relates to food, anti-obesity agent and constipation alleviator each containing the composition.

### BACKGROUND ART

Currently, in accordance with the variety of dietary life and so forth, obesity becomes a social issue. In the United States, about 65% of citizens weigh at least 13 kg higher than the healthy weight in the status quo. The annual medical expenditure due to obesity in the United States is estimated about eight trillion yens. Also in our nation, there is recently a tendency to westernize dietary life, or take a large amount of fatty food. In addition, there is a tendency to cause increases in obesity due to chronic insufficient exercises and irregular lives. Obesity is a risk factor in lifestyle-related diseases. Diseases caused easily in complication with obesity may include diabetes, hypercholesterolemia, hypertension, cardiac disease, arteriosclerosis, large intestine cancer, and gout.

Obesity is thus a critical issue in the modern society and accordingly various items have been developed for anti-obesity and diets. For example, water-soluble polysaccharides swellable in Japanese Pharmacopoeia first fluid or Japanese Pharmacopoeia second fluid and water, such as psyllimum seed gum and conjak mannan (glucomannan), may be dosed with water. In this case, the water-soluble polysaccharide swells in the stomach and intestine and provides feeling of fullness to reduce the total calorie intake.

As a factor of obesity, constipation also becomes a large issue for the modern people. Constipation causes stresses on the abdomen, and food stays in the intestine for long term such that sugars and lipids may be excessively absorbed to cause obesity. In addition, constipation facilitates production of tumor initiators from harmful bacterium in the intestine and increases the risk of large intestine cancer. To make an improvement, intake of dietary fiber called the sixth nutritional element is recommended. Intake of dietary fiber leads to absorption of water to increase the volume of feces and results in a shortened digestive passage time to eliminate constipation, preventing large intestine cancer in its turn. Accordingly, for the purpose of eliminating constipation, it is required to take dietary fiber such as polydextrose, indigestible dextrin, low-molecular weight alginic acid, and wheat bran. For example, Patent Document 1 describes a dietary fiber-containing composition composed of alginic acid and polydextrose. Patent Document 2 describes a constipation alleviator including water-soluble dietary fiber and sugar alcohol as effective ingredients. Patent Document 3 describes a constipation alleviator including water-soluble dietary fiber, insoluble fiber and vegetable juice as effective ingredients.

Patent Document 1: JP 11-75777A

Patent Document 2: JP 2000-60487A

Patent Document 3: JP 2003-12537A

### DISCLOSURE OF THE INVENTION

### Problem to be Solved by the Invention

Water-soluble polysaccharides swellable in Japanese Pharmacopoeia first fluid or Japanese Pharmacopoeia second fluid and water, such as psyllimum seed gum and conjak mannan (glucomannan), are polysaccharides derived from natural materials. Accordingly, the molecular weight and purity thereof causes large variations in the degree of water-absorption and makes it difficult to control the degree of swell. In addition, those described in the above Patent Documents are mainly for the purpose of alleviating constipation and not for controlling the degree of swell of water-soluble polysaccharides. Therefore, subjects may take them excessively to obtain a feeling of fullness and leaves a problem associated with a load imposed on the stomach and intestine.

Polydextrose, indigestible dextrin, and low-molecular weight alginic acid taken for the purpose of alleviating constipation are low-viscosity aqueous solutions. Because wheat bran and cellulose have a small amount of water absorption, they can not act fully in increasing the volume of feces on intake of dietary fiber. Further, dose of a cathartic for the purpose of alleviating constipation may soften feces and cause diarrhea. Thus it imposes a heavy load on the taker.

The present invention has an object to provide a composition containing a swelling agent that less burdens the stomach and intestine even after excessive intake, as well as to provide food, anti-obesity agent and constipation alleviator each containing the composition.

### Means to Solve the Problem

To achieve the above object, the Inventors have intensively studied many times and consequently found the following. A swelling agent composed of a water-soluble polysaccharide swellable in Japanese Pharmacopoeia first fluid or Japanese Pharmacopoeia second fluid and water, and a swell inhibitor operative to control the swell of the swelling agent are mixed in the state of solution, unified and then dried. The mixed composition makes it possible to control swell and to relieve the load imposed on the stomach and intestine even after excessive intake. Thus, the present invention provides a composition, which comprises one or more of swelling agent composed of water-soluble polysaccharides swellable in Japanese Pharmacopoeia first fluid or Japanese Pharmacopoeia second fluid and water; and one or more of swell inhibitors operative to control the swell of the swelling agent, wherein the swelling agent and the swell inhibitors are mixed in the state of solution, unified and then dried.

When the swelling agent and the swell inhibitor are mixed in the state of solution, unified and then dried, they are mixed at a molecular level in a state that can control swell of the swelling agent. Such the control may be performed by heating and dissolving the agents at the same time to unify them as the same mixed solution, followed by drying such as spray drying, drum drying, freeze drying, vacuum drying, and continuous vacuum belt drying.

In the composition according to the present invention, the swelling agent preferably includes one or more of water-soluble polysaccharide capable of dissolving to retain water and express viscosity while absorbing water and swelling at body temperature. Preferably, a constituent of the swell inhibitor can dissolve at a temperature higher than the body temperature but can not dissolve at the body temperature and performs limited water absorption. The above-described state mixed at the molecular level is made by once heating the swell inhibitor up to a dissolution temperature, that is higher than the body temperature to dissolve the swell inhibitor, and then mixed with the swelling agent dissolved at the body temperature to create a state of mixture of molecules, followed by dehydration.

The present invention also provides a food, which contain such the composition. It further provides an anti-obesity agent and a constipation alleviator, which contain such the composition as the principal ingredient. In addition, dose of a cathartic in addition to the constipation alleviator may be an effective means. When the constipation alleviator according to the present invention is added to the cathartic, the constipation alleviator increases the volume of feces and turns the muddy feces softened by the cathartic into the state of smooth softened feces. This relieves the load on a constipated person and improves QOL.

### EFFECT OF THE INVENTION

As described above, the present invention is possible to provide a composition containing a swelling agent that less burdens the stomach and intestine even after excessive intake, as well as food, anti-obesity agent and constipation alleviator each containing the composition.

### THE BEST MODE FOR CARRYING OUT THE INVENTION

In the composition containing the swelling agent according to the present invention and the food and anti-obesity agent containing the composition, the swell inhibitor is preferably configured to suppress swell of the swelling agent due to gelling the inhibitor. Preferably, such a gelling agent may contain at least one or more of agar, carrageenan, furcellaran, gellan gum, alginic acid and alginates. The swell inhibitor in the present invention is adjusted to control the swell of the swelling agent within 150 folds, preferably within 80 folds. In the composition containing the swelling agent according to the present invention and the food and anti-obesity agent containing the composition, the swelling agent may contain at least one or more of psyllimum seed gum, conjak mannan (glucomannan), xanthan gum, tamarind gum, cellulosic derivatives, tara gum and guar gum.

Preferably, the swelling agent and the swell inhibitor have a combination ratio of 95:5 to 10:90, more preferably 90:10 to 30:70.

Examples of the cathartic for use in combination with the constipation alleviator according to the present invention may include saline cathartics such as magnesium oxide, magnesium citrate, magnesium carbonate, sodium sulfate, magnesium sulfate, and magnesium hydroxide; and stimulus cathartics such as bisacodyl, sodium picosulfate, sennoide, cassia angustifolia, rheum rhaponticum, glycerin, aloe, and D-sorbitol.

### EXAMPLES

A swell inhibitor composed of agar (Ina Agar S-8 available from Ina Food Industry Co. Ltd.) is mixed with each swelling agent composed of psyllimum seed gum (Healthy Gum available from Dainippon Pharma Co. Ltd.), xanthan gum (Echo Gum available from Dainippon Pharma Co. Ltd.), conjak mannan (Mannan 100 available from Ina Food Industry Co. Ltd.) or guar gum (GR-10 available from Ina Food Industry Co. Ltd.) in mixing ratios shown in Tables 1-4, uniformly dissolved in hot water, and then dried by a drum dryer (available from Kusunoki Seisakusho Co. Ltd.) to yield compositions according to Examples 1-20. The compositions according to Examples 1-20 are powders in the state of mixture of molecules. Comparative examples of agar, psyllimum seed gum, xanthan gum, conjak mannan and guar gum were also prepared as shown in Tables 1-4.

TABLE 1

| | | Psyllimum |
|---|---|---|
| | Agar | Seed Gum |
| Comparative | 100 | 0 |
| example 1 | | |
| Example 1 | 90 | 10 |
| Example 2 | 70 | 30 |
| Example 3 | 50 | 50 |
| Example 4 | 30 | 70 |
| Example 5 | 10 | 90 |
| Comparative | 0 | 100 |
| example 2 | | |

(Weight %)

TABLE 2

| | Agar | Xanthan Gum |
|---|---|---|
| Comparative | 100 | 0 |
| example 3 | | |
| Example 6 | 90 | 10 |
| Example 7 | 70 | 30 |
| Example 8 | 50 | 50 |
| Example 9 | 30 | 70 |
| Example 10 | 10 | 90 |
| Comparative | 0 | 100 |
| example 4 | | |

(Weight %)

TABLE 3

| | Agar | Conjak Mannan |
|---|---|---|
| Comparative | 100 | 0 |
| example 5 | | |
| Example 11 | 90 | 10 |
| Example 12 | 70 | 30 |
| Example 13 | 50 | 50 |
| Example 14 | 30 | 70 |
| Example 15 | 10 | 90 |
| Comparative | 0 | 100 |
| example 6 | | |

(Weight %)

TABLE 4

| | Agar | Guar Gum |
|---|---|---|
| Comparative | 100 | 0 |
| example 7 | | |
| Example 16 | 90 | 10 |
| Example 17 | 70 | 30 |
| Example 18 | 50 | 50 |
| Example 19 | 30 | 70 |
| Example 20 | 10 | 90 |
| Comparative | 0 | 100 |
| example 8 | | |

(Weight %)

The compositions according to Examples 1-20 and Comparative examples 1-8 were subjected to measurements of degrees of swell in water, gastric juice and intestinal juice. The measurements of degrees of swell were performed by applying the absorbance ratio method of Japanese Pharmacopoeia; dispersing each 3g of the compositions according to Examples 1-20 and Comparative examples 1-8 in water, Japanese Pharmacopoeia first fluid and Japanese Pharmacopoeia second fluid; and measuring the quantity of filtrate (g) when filtered using glass wool. The measured value was defined as a degree of swell, which is a water absorbency magnification per 1 g of the composition, and calculated by Equation 1. The results are shown in Tables 5-8.

Degree of Swell=(100g−Quantity of Filtrate(g))/3g [Equation 1]

TABLE 5

| | Degree of | Degree of | Degree of |
|---|---|---|---|
| | Swell | Swell | Swell |
| | (Water) | (First Fluid) | (Second Fluid) |
| Comparative | 8 | 10 | 8 |
| example 1 | | | |
| Example 1 | 10 | 12 | 10 |
| Example 2 | 30 | 25 | 25 |
| Example 3 | 43 | 35 | 37 |
| Example 4 | 65 | 45 | 50 |
| Example 5 | 160 | 123 | 125 |
| Comparative | 167 | 128 | 130 |
| example 2 | | | |

TABLE 6

| | Degree of | Degree of | Degree of |
|---|---|---|---|
| | Swell | Swell | Swell |
| | (Water) | (First Fluid) | (Second Fluid) |
| Comparative | 8 | 10 | 8 |
| example 3 | | | |
| Example 6 | 20 | 18 | 19 |
| Example 7 | 110 | 80 | 90 |
| Example 8 | 140 | 100 | 103 |
| Example 9 | 180 | 130 | 140 |
| Example 10 | 270 | 170 | 215 |
| Comparative | 286 | 175 | 228 |
| example 4 | | | |

TABLE 7

| | Degree of | Degree of | Degree of |
|---|---|---|---|
| | Swell | Swell | Swell |
| | (Water) | (First Fluid) | (Second Fluid) |
| Comparative | 8 | 10 | 8 |
| example 5 | | | |
| Example 11 | 13 | 13 | 14 |
| Example 12 | 53 | 52 | 48 |
| Example 13 | 65 | 64 | 70 |
| Example 14 | 80 | 77 | 75 |
| Example 15 | 130 | 104 | 90 |
| Comparative | 134 | 109 | 94 |
| example 6 | | | |

TABLE 8

| | Degree of | Degree of | Degree of |
|---|---|---|---|
| | Swell | Swell | Swell |
| | (Water) | (First Fluid) | (Second Fluid) |
| Comparative | 8 | 10 | 8 |
| example 7 | | | |
| Example 16 | 16 | 14 | 18 |
| Example 17 | 75 | 70 | 68 |
| Example 18 | 89 | 80 | 87 |
| Example 19 | 110 | 120 | 105 |
| Example 20 | 165 | 170 | 185 |
| Comparative | 170 | 178 | 192 |
| example 8 | | | |

As obvious from Tables 5-8, intake of only the swell inhibitor according to Comparative example 1, 3, 5 and 7 results in a smaller degree of swell and less effects on anti-obesity and constipation alleviation. In contrast, intake of only the swelling agent according to Comparative example 2, 4, 6 and 8 results in a larger degree of swell and heavily burdens the stomach and intestine. As for the compositions according to Examples 1-20, desired degrees of swell can be achieved with variations in the mixing ratio. Thus, it is possible to exert appropriate effects on anti-obesity and constipation alleviation in accordance with takers.

1. A composition, comprising: one or more of swelling agents composed of water-soluble polysaccharide swellable in Japanese Pharmacopoeia first fluid or Japanese Pharmacopoeia second fluid and water; and one or more of swell inhibitors operative to control the swell of the swelling agents, wherein the composition are produced by that the swelling agent and the swell inhibitor are mixed in the state of solution, unified and then dried.

2. The composition according to claim 1, wherein the swelling agent and the swell inhibitor have a combination ratio of 95:5 to 10:90.

3. A food, comprising the composition according to claim 1 or 2.

4. An anti-obesity agent, comprising the composition according to claim 1 or 2 as the principal ingredient.

5. A constipation alleviator, comprising the composition according to claim 1 or 2 as the principal ingredient.

6. A medicament, comprising the constipation alleviator according to claim 5 in combination with an aperient.

7. A process for preventing obesity comprising ingesting a composition, said composition comprising:
a swelling agent comprising a water-soluble polysaccharide swellable in water and at least one of Japanese Pharmacopoeia first fluid and Japanese Pharmacopoeia second; and
a swell inhibitor operative to control the swell of the swelling agent;
said composition formed by dissolving the swelling agent and the swell inhibitor to form a solution and drying the solution.

8. The process of claim 7, wherein the swelling agent and the swell inhibitor have a combination ratio of 95:5 to 10:90

9. A process for alleviating constipation comprising ingesting a composition, said composition comprising:
a swelling agent comprising a water-soluble polysaccharide swellable in water and at least one of Japanese Pharmacopoeia first fluid and Japanese Pharmacopoeia second; and
a swell inhibitor operative to control the swell of the swelling agent;
said composition formed by dissolving the swelling agent and the swell inhibitor to form a solution and drying the solution.

10. The process of claim 9, wherein said composition further comprises an aperient.

11. The process of claim 9, wherein the swelling agent and the swell inhibitor have a combination ratio of 95:5 to 10:90.

12. The process of claim 11, wherein said composition further comprises an aperient.
